(19) United States
(12) Patent Application Publication
(10) Pub. No.: US 2002/0026157 A1
(43) Pub. Date: Feb. 28, 2002

(54) Feminine, self-administrable sampling and topical therapy device and method

(76) Inventor: Arthur M. Fournier, Miami, FL (US)

Correspondence Address: MALLOY & MALLOY, P.A., 2800 S.W. Third Avenue, Historic Coral Way, Miami, FL, 33129, US

(21) Appl. No.: 09/955,557

(22) Filed: Sep. 18, 2001

Related U.S. Application Data
(63) Continuation-in-part of application No. 09/867,337, filed on May 29, 2001.
Application No. 09/867,337 is a continuation-in-part of application No. 09/716,648, filed on Nov. 20, 2000.
Application No. 09/716,648 is a continuation-in-part of application No. 09/206,760, filed on Dec. 7, 1998, now Pat. No. 6,155,990.

(51) Int. Cl. A61M 5/00; A61M 31/00

(52) U.S. Cl. 604/279; 604/187

(57) ABSTRACT

A female self-administrable topical therapy device is disclosed which can be self administered by women to collect specimens from the vaginal cavity and/or apply topical treatment therapy therein. In particular, the device includes an insertion conduit structured to be at least partially introduced into the vaginal cavity of the female and an administration element housed within the insertion conduit to ultimately apply a treatment material. A handle coupled with the administration element is provided to protrude and preferably release the administration element from the insertion conduit and cause it to engage an affected location within the vaginal cavity in order to effectively apply the topical therapy thereto.

### BACKGROUND OF THE INVENTION

The present application is a continuation-in-part application of previously filed, now pending application having Ser. No. 09/867,337 filed on May 29, 2001 which is a continuation-in-part application of previously filed, now pending application having Ser. No. 09/716,648 filed on Nov. 20, 2000, which is a continuation-in-part of issued U.S. Pat. No. 6,155,990, issued Dec. 5, 2000, the contents of both of which are incorporated herein by reference.

### CLAIM OF PRIORITY

1. Field of the Invention

Screening for cervical cancer in women using cytological techniques, as well as the screening for various other vaginal related diseases, conditions and infections has been possible for many years, the present invention, however, relates to a self-sampling device which is easy and convenient to utilize by a women herself, without direct viewing requirements, such that the woman can effectively obtain a sample to be tested in privacy and with minimal discomfort. Moreover, the present device and method allow for a single sampling to be performed and contained in a manner that allows for a variety of different test procedures to be implemented in order to screen for the presence of many different conditions, including cytology, micro-array assays and tumor markers. As yet a further advantage, the present invention permits a user to self-administer topical agents, such as via direct contact and/or a suppository, thereby permitting discrete, safe and effective utilization of topical therapies directly by the patient.

2. Description of the Related Art

In spite of the progress that has been made over the years in connection with the detection, testing and treatment for various illnesses, infections and other conditions associated with the vagina and/or cervix of a woman, there still remain a number of problems with the present technology. For example, conventional pap tests show a high percentage of smears of undetermined significance that requires further testing. This problem has led to the development of “thin prep” technology. Thin prep technology requires that cells be immersed in fixative and centrifuged prior to analysis. Other advances in diagnostic technology are the discovery of DNA probes for human papilloma virus (HPV), the causative agent of cervical cancer, and for chlamydia, a common infection in women. Test for HPV may soon replace conventional pap smears as the initial screening test for cervical cancer.

Unfortunately, given the present technology, collection of specimens, such as cytology specimens to screen for cancer, currently requires a speculum examination. Such an Examination, which is frequently uncomfortable and embarrassing for women, requires direct viewing into the vaginal cavity, which for purposes of this application is intended to include the vagina, cervix, and surrounding areas, and as such must be performed by a physician or nurse practitioner, thus also adding to the expense and inconvenience. In such circumstances the specimen obtained is applied directly to a glass slide, which is not compatible with automated cytologic analysis or necessary for HPV assay. The same problems of discomfort, embarrassment, expense and processing also apply to the obtaining of specimens to diagnose vaginal infections such as candidiasis, gonorrhea, human papilloma virus and chlamydia.

In an attempt to minimize the problems associated with traditional sampling techniques, a number of improved sampling devices were developed. Unfortunately, most such sampling devices were designed prior to the invention of thin prep and HPV assay technologies, and are generally of minimal if any effect for such testing, or were designed specifically to obtain a specimen in the setting of a conventional speculum examination, which does not alleviate some of the primary problems associated with speculum testing. Given these problems, there is still a need for an improved, inexpensive self sampling device which asymptomatic or symptomatic women can use in the privacy of their home at any time, that is adaptable to automated cytology methods (thin smear), HPV assay and microbial culture, and does not require direct viewing or third party assistance to obtain an effective and multi-purpose sample capable of aiding in the detection of a variety of different infections, illnesses, conditions, etc. Furthermore, there is a significant need for a device, which when a condition has been identified, can be used in the safe, convenient and effective treatment of the condition by the patient. For example, it is often the case that topical immuno-modular therapy can be employed directly at an effected area, however, present techniques and devices require medical and/or direct vision application of necessary medications and treatments. As such, it would be beneficial to provide a device and method which can by used by a woman in privacy, but which will still safely and effectively deliver the necessary medication to the desired delivery site without direct viewing.

### SUMMARY OF THE INVENTION

Accordingly, the present invention is directed towards feminine self-administrable sampling and topical therapy device that will allow for the self sampling of cervical or vaginal specimens from the interior of the vaginal cavity, without direct viewing, and can also be configured to permit the direct application of a topical therapeutic agent, such as in the form of a suppository. The device is preferably inexpensive to produce, easy to destroy after use, and easy for women to use to perform a variety of testing and topical therapies, all with the single device and with a single specimen collection procedure. Furthermore, a preferred embodiment of the present device allows for the safe transportation of either solid of fluid specimens to laboratories for analysis, and is easily adaptable for centrifugation and thin-film preparation, or DNA probe for HPV, which is a technological improvement over the older direct preparation from a smear. Moreover, the present device may also be used to obtain specimens for culture or microbiologic assay, thus making it useful in the diagnosis of vaginal infections and for epidemiologic studies of sexually transmitted diseases. Indeed, the device and method offer advantages over brushes in the setting of direct visualization of the cervix, particularly when the patient is pregnant.

In an embodiment wherein topical therapy is achieved utilizing the present device, easy positioning and delivery of a topical agent, such as in the form of a suppository can be effectively delivered at or very near a treatment area, and effectively released for ultimate interaction with the patient even after complete removal of a remainder of the device.

These and other features and advantages of the present invention will become more clear when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a perspective view of a self sampling embodiment of the present invention taken from one side and below.

FIG. 2 is a cross-sectional view demonstrating the internal design.

FIG. 3 is a diagram demonstrating how the device is designed to fit and seal a standard cylindrical tube, in order to process the specimen using thin-film cytology.

FIG. 4 is an alternative embodiment including a it collapsible handle.

FIG. 5 is an alternative embodiment of the present invention including a removable collection element.

FIG. 6 is a side view of still another embodiment of the self-administrable sampling and topical therapy device of the present invention in use for the administration of a suppository.

Like reference numerals refer to like parts throughout the several views of the drawings.

### DETAILED DESCRIPTION OF THE PREFERRED EMBODIMENT

Please refer to FIGS. 1-6 for a complete understanding of this invention. The drawings represented the current best conceptualizations of the design of the feminine self-administrable sampling and topical therapy device, and are not to be construed as a restricting or limitation of its scope.

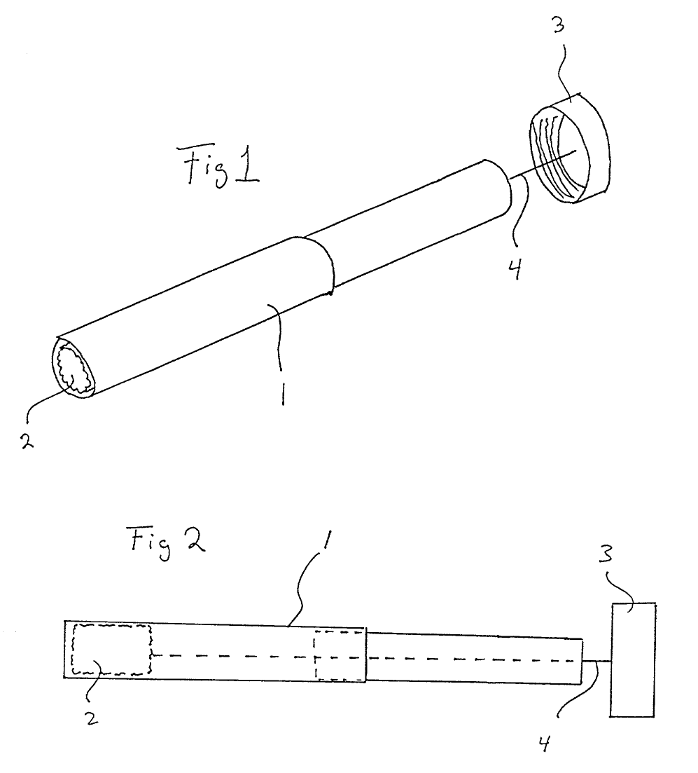

As can be seen in the embodiment of FIGS. 1 and 2, the device may be considered an assembled unit comprising sub-units such as: a) an insertion conduit, such as the illustrated “telescoping” cylindrical tube (1) which houses, b) the specimen-gathering collection element in the case of a self-sampling embodiment, such as a sponge (2). The sponge is preferably secured to an elongate handle (4), that is capable of extending the collection element (2) into the vaginal cavity along a path defined and guided by the insertion conduit (1). Also as shown in the embodiment of FIGS. 1-4, a cap (3) may be provided and may define an exterior portion of the handle (4). The cap may further function to shield and later contain the collection element (2), as will be described.

Looking in further detail at the insertion conduit (1), it may be configured as a cylindrical sheath constructed of medical-quality cardboard and coated with wax or Teflon to allow for minimal friction on insertion. Furthermore, although it is contemplated that a single tube segment of sufficient length may be provided, in the illustrated embodiment, so as to achieve a more compact configuration, the telescoping type tube configuration is preferably employed. In either embodiment, the insertion conduit functions to shield and protect the collection element during insertion. For example, although it is contemplated that the insertion conduit (1) is first introduced into the vaginal cavity, and subsequently, the collection element (2) is passed therethrough, in the illustrated embodiment, the collection element (2) is contained by the insertion conduit (1) as the complete unit is inserted and extended into the vaginal cavity. For example, with the “telescope” fully extended and the collection element (2) may be housed in the anterior chamber of the insertion conduit (1) during insertion, thus acting as a sheath to protect the collection element.

Looking further at the insertion conduit (1), it is preferably sized and configured so as to define a path for the collection element (2), and thereby guide the collection element (2) to a desired sample collection location or medication application location. By effectively sizing the insertion conduit to correspond the average insertion depth to reach the cervix (typically about 13 cm), the insertion conduit (1) maybe inserted into the vaginal cavity until resistance is met or until a predefined marking on the conduit itself. As a result, a user does not have to directly visualize the interior of the vaginal cavity, and the collection element (2) is guided directly to the sample collection location without undue manipulation that may lead to injury or discomfort.

With the insertion conduit (1) in place and effectively positioned to guide the collection element (2), the collection element (2) may then be extended therefrom by the handle (4). For example, by grasping the posterior end of the insertion conduit (1) between two fingers and pressing on the protruding portion of the handle (3), the collection element (2) will extend into the vaginal vault, contacting the sample collection location.

Looking further to the handle (4) and as illustrated in FIG. 4, the handle itself may also be collapsible, either into its own sheath, as in the illustrated embodiment, and/or into the outer portion of the insertion conduit (1). Such an embodiment may be especially beneficial for effective packaging and/or for the introduction of both the handle (4) and the collection element (2) into a fixative container of a smaller size.

The collection element (2) may be composed of several possible flexible, perhaps resilient materials, including, but not limited to cellulose, natural or artificial sponge material. As such, the collection element (2) preferably includes an at least, mildly abrasive quality that serves to exfoliate the collection location and dislodge cells to be gathered as part of the collected sample. Furthermore, the collection element (2) may have good adherent and mildly abrasive qualities, and in such an embodiment, the vacuum created by the sponge upon insertion should draw cells down from the cervical canal without the trauma that might be created by a traditional spatula. It also obviates the need for direct visualization of the cervical os. Of course, it is understood that the composition of the collection element may be modified under special circumstances; i.e., a more abrasive sponge may be warranted in screening for HIV inter-menses, and a more absorptive sponge is indicated during menses or to obtain specimens during abnormal uterine bleeding.

Looking in further detail to the collection of the specimen, after insertion of the insertion conduit (1) and extension of the collection element (2), the handle (4) may be rotated to exfoliate the surface and obtain the specimen. The collection element (2) may then be retracted in order to return it to the anterior chamber of the insertion conduit (1) prior to removal. In this manner the collection element (2) is shielded and the specimen preserved on the collection element as it is removed from the vaginal cavity. In the case of a telescoping insertion conduit, a rim on the inner tube and a groove on the inner surface of the outer tube may be provided to assure that the telescope mechanism does not collapse prior to the collection element returning to the anterior chamber.

In this preferred, illustrated embodiment, the overall length of the device may be approximately 15 cm, the insertion conduit (1) being about 13 cm when fully extended and disposed in an extended orientation. As such, the handle (4) which may be in the range of 8-18 cm long, and as indicated may be of a fixed dimension or extendable, is preferably about 14 cm when extended so as to provide for sufficient protrusion of the collection element from the insertion conduit for engagement with the patient. Moreover, in the illustrated embodiment, the width or diameter will be about 1.5 cm. These dimensions will accommodate the standard vaginal cavity depth of 12.7 cm for a mature woman, and are sufficiently narrow to allow for comfortable insertion. In the telescoping embodiment, the device will collapse to about 7 cm, so as to define a compressed orientation, allowing it to be easily inserted into a specimen tube (5) of about 11.5 cm in depth. As will be described, as an alternative, the collection element may be expulsed by itself into the fixative.

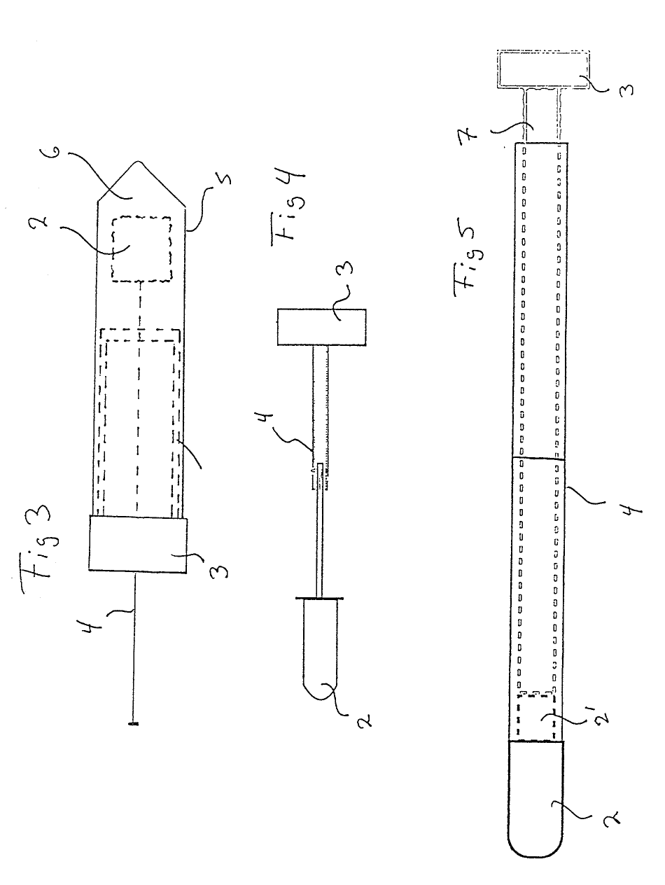

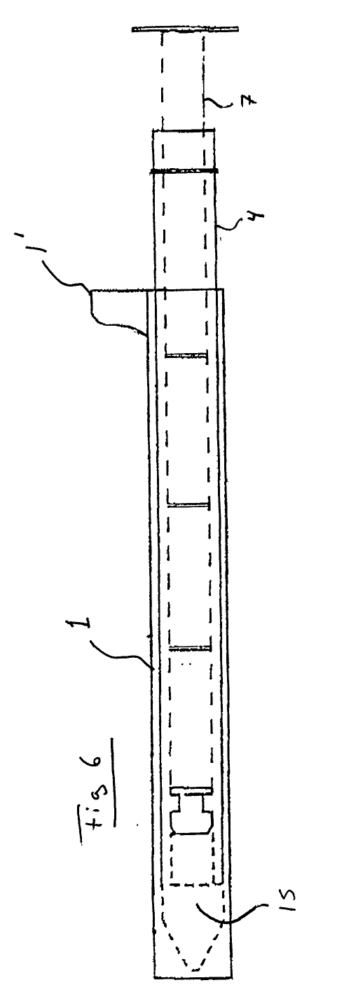

In particular, the present invention also preferably includes a fixative container (5) into which the collection element (2), with the sample contained thereon, is introduced. As will be described in further detail, the fixative container (5) includes a fixative (6) therein to preserve the specimen and into which the collection element may be immersed. In the illustrated embodiment, the entire collection portion of the device may be introduced into the fixative container (5), the collapsed insertion conduit (1) and/or handle (4) allowing for complete introduction thereof with the collection element exposed. Moreover, in the embodiment wherein the exterior portion (3) of the handle (4) is in the form of a screw on cap, the cap may be configured to form a tight seal on the fixative container (5). Also in such an embodiment, if the handle (4) is of a fixed size or a large collapsed size, it may extend out through the cap (see FIG. 3), its length of protrusion determined by the collapse of the telescope and the need for the collection element sponge to be freely immersed in the fixative (6). Of course, as an alternative embodiment, and as illustrated in FIG. 5, the collection element may be configured to be removable from the handle (4),and as such, once removed from the patient, the collection element may be placed inside the fixative container (5) separate from the handle (4) and remaining structure. In such an embodiment, a plunger element (7) may be provided and configured to be pushed by the user after collection of the sample so as to detatch the collection element (2) from the handle (4). As shown in FIG. 5, in such an embodiment the collection element may have an attachment segment (2′) that extends into the handle (4) so as to maintain secure engagement until removed, either directly by the user or by pushing of the plunger element (7). The collection element (2) may then be sealed in the fixative container by a conventional cap and a remainder of the structure discarded or sterilized for re-use.

As mentioned, the collection element (2) is preferably immediately immersed in the fixative after removal of the device from the patient, avoiding problems associated with drying. This fixative thereby preserves and maintains the collected specimen, allowing it to be maintained and/or transported for testing of the specimen. In this regard, home testing and/or transport to a laboratory for testing may be achieved. In either case, when testing is desired, the fixative container (5) is preferably agitated to liberate cells and/or fluid from the sponge, centrifuged and the supernatant discarded. The cellular contents may be removed and prepared as a thin-prep slide for either manual or automated cytologic examination using conventional cytologic techniques. As to fluid specimens, a culture test may be performed, and/or a molecular pathology assay type test may be performed. These include “wet mount”, KOH prep for fungal examinations or PCR assays for chlamydia and human papilloma virus, or other infectious agents such as human immunodeficiency and hepatitis viruses and tumor markers from a variety of cancers. The device can then be disposed of using “universal precautions” and incinerated. It should be noted that both the insertion conduit and the collection element may be used to obtain vaginal as well as cervical samples form what is generally defined as the vaginal cavity.

Looking in further detail to the fixative fluid (6) it is preferably configured to be both fluid and cell compatible so as to facilitate cytology and culture/micro-array assay testing from the same collected specimen, or from specimens collected utilizing the same techniques. As such, the fixative fluid may be said to be RNA friendly, and may comprise a fluid varying from 5% alcohol, to the commercially available saccomanno fluid that is composed of ethyl alcohol, isopropyl alcohol, methyl alcohol, polyethylene alcohol and MIBK.

Accordingly, although designed for self-sampling of cytologic specimens, it may have wide applications in the diagnosis of vaginal infections, in conducting epidemiologic studies of sexually transmitted diseases or in comparing the utility of PCR assay for human papilloma virus to conventional cytology as a screening test for cervical cancer. Indeed, the device may also be used for cell sampling of colon, oral, uterine and dermal tissues with only minor modifications, and if desired, processed in such a way as to allow for assay of cytology, infectious agents and tumor markers associated with oral, rectal and uterine cancer, among other possible conditions. As yet another alternative, the present device and method may also be utilized to brush or spatula to obtain specimens under direct vision. This application has special merit in the case of pregnant women, in whom brushing is contradicted. Finally, the insertion conduit (1) may be used for culture, micro-array assay or other tests to diagnose vaginal infections. Further, the present invention may also be utilized as an applicator of topical treatments and medications. For example, direct topical application of medication directly on the cervix has generally been found to be difficult due to the lack of an appropriate instrument that not only effectively reaches the application site, but also maintains the medication to be applied in a generally shielded state during inter-labial introduction. As such, it is recognized that the medication could be effectively employed by applying the medication to and administration element defined by the collection element (2), inserting the shielded collection element into the vaginal cavity via the protective insertion conduit (1), and extending the collection element (2) into contact with the cervix or other treatment region so as to directly apply the medication.

Turning to the embodiment of FIG. 6, however, the administration element may be defined by a suppository type element (15). The suppository element (15) can be effectively disposed and removably retained at an end of the handle (4), for subsequent discharge within the vaginal cavity. For example, the plunger element (7) may extend through the handle (4) and effectively push out the suppository element (15) once inside the vaginal cavity. As such, proper positioning and administration of the medication or other treatment material is ensured. Also, in such an embodiment, the exterior sheath (1) may be a single element or multiple telescoping elements, the preferred configuration being such that the suppository element (15) or other application device is shielded and the medication is not prematurely released and/or applied prior to entry into the appropriate region of the vaginal cavity. Moreover, the exterior sheath (1), may, if desired, include a tab element (1′) disposed thereon. The tab element (1′) may be configured and disposed to permit the sheath (1) to be at least partially retracted from over the suppository element (15) so as to allow its effective release, and/or may provide leverage against which the plunger element (7) is pushed for discharge of the suppository (15). In this manner, safe and accurate depositing and administration of the suppository for topical therapy can be achieved directly by the patient.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. A feminine self-administrable topical therapy device comprising:
an insertion conduit, said insertion conduit structured to define an access path into a vaginal cavity of a female;
an administration element at least temporarily housed within said tube,
a handle coupled with said administration element and structured to protrude said administration element from said insertion conduit;
said insertion conduit further structured to guide said administration element towards a location within the vaginal cavity of the female so as to eliminate a direct visualization requirement; and
said administration element structured to engage the sample location and administer a treatment thereon.

2. The feminine self-administrable topical therapy device recited in claim 1 wherein said handle is further structured to retract said administration element into said insertion conduit for protected removal from the vaginal cavity.

3. The feminine self-administrable topical therapy device recited in claim 1 wherein said handle is structured to protrude said administration element from said insertion conduit by maintaining said administration element in place during at least partial retraction of said insertion conduit.

4. The feminine self-administrable topical therapy device recited in claim 1 wherein said administration element includes a suppository comprised at least partially of said treatment material and structured to be removably secured to said handle so as to be released within said vaginal cavity.

5. The feminine self-administrable topical therapy device recited in claim 4 wherein said handle includes a plunger assembly structured to separate said administration element from said handle.

6. The feminine self-administrable topical therapy device recited in claim 1 wherein said handle is structured to extend said administration element approximately 8-18 cm into said vaginal cavity.

7. The feminine self-administrable topical therapy device recited in claim 1 wherein said insertion conduit includes a telescoping tube structured to extend into said vaginal cavity.

8. The feminine self-administrable topical therapy device recited in claim 1 wherein said handle includes a telescoping configuration.

9. A feminine self-administrable topical therapy device comprising:
an insertion conduit, said insertion conduit structured to be at least partially introduced into a vaginal cavity of a female;
an administration element at least temporarily housed within said insertion conduit;
a handle coupled with said administration element and structured to extend said administration element from said insertion conduit; and
said administration element structured to be released within the vaginal cavity.

10. A method of obtaining administering a topical treatment material within a vaginal cavity of a female comprising:
the female introducing an insertion conduit at least partially into the vaginal cavity;
the female extending an administration element from the introduced insertion conduit into the vaginal cavity without direct viewing and along a path guided by said insertion conduit;
the female engaging a cervix within the vaginal cavity with the administration element and affirmatively applying a treatment material; and
the female removing said handle and said insertion conduit.

11. The method of claim 10 wherein the female engaging the cervix within the vaginal cavity with the administration element further comprises releasing the administration element in the form of a suppository from the handle in a vicinity of the cervix.

12. The method of claim 10 further comprising the step of removing said administration element after applying said treatment material.
